(19) United States
(12) Patent Application Publication
(10) Pub. No.: US 2011/0201128 A1
(43) Pub. Date: Aug. 18, 2011

(54) BLOOD MARKERS OF TRANSPLANTED INTESTINE REJECTION

(75) Inventors: Thierry Yandza, Nice (FR); Nathalie Neveux, Paris (FR); Luc Cynober, Sceaux (FR)

(73) Assignee: CENTRE NATIONAL DE LA RECHERCHE SCIENTIFIQUE- CNRS, PARIS CEDEX 16 (FR)

(21) Appl. No.: 12/744,795

(22) PCT Filed: Nov. 27, 2008
(86) PCT No.: PCT/FR08/01647

§ 371 Date: Sep. 30, 2010

(30) Foreign Application Priority Data
Nov. 28, 2007 (FR) 0708317

(51) Int. Cl. G01N 33/53; G01N 33/68; G01N 30/02

(52) U.S. Cl. 436/501; 436/86; 73/61.52; 73/23.35

(57) ABSTRACT

The present invention relates to a method for detecting bowel transplant rejection.

The detection method according to the invention is a method for the in vitro detection of bowel transplant rejection comprising steps for measuring the concentration at markers in a fluid sample, calculating the difference for each marker with a reference concentration, comparing for each marker the difference with a discriminatory threshold variation and assigning a score.

The present invention is particularly applicable in the medical field, more specifically in the field of transplantations and transplant rejection detection.

### FIELD OF THE INVENTION

The present invention relates to a method for detecting bowel transplant rejection.

The present invention is particularly applicable in the medical field, more specifically in the field of transplantations and transplant rejection detection.

In the description hereinafter, the references between square brackets ([ ]) refer to the list of references given after the examples.

### PRIOR ART

There are two primary causes for irreversible chronic bowel failure: a short anatomical small bowel following small bowel resections for example or a short functional small bowel due to bowel cell disease or bowel motility disorders (motor dysfunction causing chronic partial obstructions and malabsorption). The main result of chronic bowel failure is a dependency on parenteral nutrition. This parenteral nutrition involves a plurality of drawbacks, primarily the material constraints of patient management and potentially life-threatening complications for patients. An alternative to parenteral nutrition and likewise a potentially life-threatening condition for patients is a bowel transplant procedure.

Bowel transplantation has seen a constant rise since 2001. Over 140 transplants are carried out in the United States each year. In France, the potential is 40 to 50 new cases each year. In this way, according to the international intestinal transplant registry, 1292 transplants were performed worldwide on 1210 patients. Of these 1210 transplant patients, in March 2005, only 658 were still alive, despite the increase in the survival rate of transplant patients. One of the primary causes of death in transplant patients after sepsis, is transplant rejection representing 9% of deaths. Furthermore, sepsis, which is responsible for 50% of deaths, generally complicates the treatment of rejection diagnosed at a late stage.

Therefore, the identification of transplant rejection markers for bowel transplants similar to those for kidney transplants or liver transplants would help prevent deaths due to transplant rejection and thus reduce the number of deaths due to transplant rejection significantly.

Prior art studies have proposed plasma citrulline as a potential marker for bowel transplant rejection. Indeed, citrulline in P. Crenn et al. “Postabsorptive plasma citrulline concentration is a marker of absorptive enterocyte mass and intestinal failure in humans. Gastroenterology” 2000; 119: 1496-505 [1] is described as marker for bowel failure and in LC. Lutgens et al. “Monitoring myeloablative therapy-induced small bowel toxicity by serum citrulline concentration: a comparison with sugar permeability test” Cancer 2005; 103: 191-9 [2] as a cell lysis marker in the bowel. In the article by P A. Pappas et al. “Serum citrulline and rejection in small bowel transplantation: a preliminary report” Transplantation 2001; 72: 1212-16 [3], the authors linked citrulline with bowel transplant rejection and in P A. Pappas et al “An analysis of the association between serum citrulline and acute rejection among 26 recipients of intestinal transplant” Am J Transplantation 2004; 4: 1124-32 [4], the authors describe citrulline as a potential marker for bowel transplant rejection.

However, clinical trials using this marker have not demonstrated the reliability of said marker. Indeed, numerous confounding and methodological factors such as age, postoperative follow-up, kidney function and surgical stress altered the results and made it impossible to confirm that plasma citrulline is a bowel transplant rejection marker.

The only current means to confirm a rejection diagnosis is to perform iterative blind biopsies with a histological examination, in the hope of diagnosing rejection early.

Early diagnosis of bowel transplant rejection improves patient management and outcome.

Therefore, there is a genuine need for reliable markers and a method for the non-invasive detection of said markers, to obtain a rapid diagnosis of bowel transplant rejection so as to detect bowel transplant rejection as early and as reliably as possible, thus enabling the reduction of the number of rejections and deaths associated with bowel transplant rejection.

### DESCRIPTION OF THE INVENTION

The aim of the present invention is specifically to respond to the abovementioned requirements and drawbacks by providing a method for detecting bowel transplant rejection.

The method according to the invention comprises the in vitro detection of bowel transplant rejection comprising the following steps:

a) measuring the concentration in a fluid sample of at least two markers selected from the group comprising: aspartate, citrulline, taurine and phenylalanine,

b) calculating the difference for each marker of said concentrations measured in step a) with a reference concentration of said marker,

c) comparing for each marker the absolute value of said difference with a discriminatory threshold variation,

d) assigning a score for each marker according to said comparison and summing said scores.

According to the invention, bowel transplant rejection detection is observed in 100% of cases when the sum of the scores calculated in step d) is greater than or equal to 2.

The present invention also relates to a kit for the implementation of the method according to the invention.

The present invention also relates to the use of the method according to the invention to diagnose bowel transplant rejection.

According to the present invention, the term “fluid sample” or “sample” refers to any fluid sample suitable for measuring the concentration of at least two markers. For example, it may consist of any biological fluid, for example, a blood sample, a plasma sample, a cerebrospinal fluid sample, a urine, saliva, sweat, synovial fluid sample, etc.

According to the present invention, the fluid sample may be obtained for example from a specimen taken on a mammal or rodent. The mammal may for example be a human being, pig and/or monkey.

According to the present invention, the fluid sample may be obtained from a transplanted or non-transplanted, healthy or ill subject, for example suffering from bowel, heart, kidney, liver, lung, pancreatic failure, etc. and/or a subject having undergone bowel transplantation.

According to the present invention, the fluid sample may also be a previously diluted, concentrated and/or purified sample. It may also consist of a solid or semi-solid sample previously treated for solubilisation, for example by dispersion in a physiological fluid.

According to the present invention, the marker concentration may be measured by any means known to those skilled in the art. For example, the concentration of at least one of the markers may be measured by a means selected in the group comprising High-Performance Liquid Chromatography (HPLC), immunoassay, reversed-phase gas chromatography with pre-column derivation with various reagents, such as 9-fluorenymethylchloroformate, phenylisothiocyanate, dimethylaminaphthalenesulphonyl chloride, dimethylaminoazobenzenesulphonyl chloride and/or 4-fluoro-7-nitrobenzo-2-oxa-1,3-diazole, ion exchange chromatography with post-column derivation using OPA and/or ninhydrin, automated ion exchange chromatography using the Hitachi L-8500A device as described in Le Boucher J, et al. Amino acid determination in biological fluids by automated ion-exchange chromatography: performance of Hitachi L-8500A. Clinical Chemistry 1997; 43:1421-28 [5].

The present invention also describes a method as defined above wherein, in step a), the concentration of 2, or 3, or 4 markers is measured.

According to the present invention, the reference concentration in step b) is a concentration for each marker determined by measuring in a sample from a subject or from a standardisation of measurements using different samples from different subjects.

According to the present invention, the abovementioned subject(s) may be for example one or a plurality of subjects not having undergone bowel transplantation, said subject(s) may be one or a plurality of healthy subjects or subject(s) waiting for a bowel transplant. For example, the subject(s) may be one or a plurality of subjects similar in age, weight, height and/or physiological characteristics to the subject from whom the sample whereon the marker concentration is measured in step a) is obtained.

According to the present invention, the reference concentration may be measured by any measurement means suitable for performing the method according to the invention. For example, the reference concentration may be measured using the abovementioned measurement methods.

In one particular embodiment of the present invention described hereinafter, the reference concentration is measured on a sample taken on a patient prior to transplantation. In this embodiment, the reference concentration for each marker is the concentration of said marker prior to transplantation.

According to the present invention, the calculation performed in step b) consists of calculating the difference for each marker of the value measured in the fluid sample in step a) with the reference value as described above.

According to the present invention, the calculation performed in step b) of the method according to the invention may be performed using any means known to those skilled in the art. For example, using a calculator, mental arithmetic, by an automated means, etc.

According to the present invention, the absolute value of the results of the calculation performed in step b) is retained for step c) of the method according to the invention.

According to the present invention, the comparison made in step c) of the method according to the invention determines whether the absolute value of the different calculated in step b) for each marker is greater than or less than a discriminatory threshold variation for each marker.

According to the present invention, the term “discriminatory threshold variation” refers to a value defined for each marker corresponding to the minimum variation of the concentration of each marker in the presence of transplant rejection. This discriminatory threshold variation may also be referred to as the “discriminatory value”.

According to the present invention, the discriminatory threshold variation may be determined by any means known to those skilled in the art. For example, the discriminatory threshold variations for each marker were determined by defining receiver operating characteristic or “ROC” curves constructed on the basis of the various thresholds studied and by establishing the threshold offering the best compromise of sensitivity (Se) and specificity (Sp): maximisation of the Youden index (Se+Sp−1).

For each amino acid, it is possible to calculate the absolute value of the difference between the reference concentration and the concentration in a fluid sample: reference value−value in sample.

For each variation threshold studied, it is possible to calculate the sensitivity, specificity and the positive and negative predictive value.

According to the present invention, the term “sensitivity” refers to the probability of the absolute value of the difference being greater than the discriminatory threshold variation if the animal belongs to the rejection group.

According to the present invention, the term “specificity” refers to the probability of the absolute value of the difference being less than or equal to discriminatory threshold variation if the animal belongs to the non-rejection group.

According to the present invention, the term “positive predictive value” refers to the probability of the animal belonging to the rejection group if the absolute value of the difference is greater than the discriminatory threshold variation.

In the present invention, the term “negative predictive value” refers to the probability of the animal belonging to the non-rejection group if the absolute value of the difference is less than or equal to the discriminatory threshold variation.

The plasma levels of the four amino acids may be significantly correlated via the sensitivity, specificity, positive predictive value and negative predictive calculations with the incidence of rejection.

The sensitivity of each marker may be determined by the probability of the absolute value of the difference being greater than the threshold if the animal belongs to the rejection group.

The specificity of each marker may be determined by the probability of the absolute value of the difference being less than or equal to the threshold if the animal belongs to the non-rejection group.

The positive predictive value of each marker may be determined by the probability of the animal belonging to the rejection group if the absolute value of the difference is greater than the discriminatory threshold variation.

The negative predictive value may be determined by the probability of the animal belonging to the non-rejection group if the absolute value of the difference is less than or equal to the discriminatory threshold variation.

According to the present invention, a logistic regression model may be constructed comprising the rejection variable as the dependent variable and the score as the independent variable as described in SAS/STAT Software: “Changes and enhancements through release 6.11”. In. 6.11 ed. Cary, N.C., USA; 1996. [6]. If Y is the response of an animal, annotated 0 in the absence of rejection and 1 in the presence of rejection, x is the independent variable vector and π is the probability of the response to be modelled, the logistic model may be expressed according to the following formula:

$$log it ( π ) ≡ log ( \frac{π}{1 - π} ) = α + {β}^{′} x$$

wherein α is the intercept and β′ is the parameter vector.

If N is the total number of observations, t is the total number of response pairs, n_{c} is the number of consistent pairs, n_{d} is the number of discrepant pairs and t−n_{c}−n_{d} is the number of equitable pairs. The area under the curve (AUC) may be calculated as follows:

C=(n_{c}+0.5(t−n_{c}−n_{d}))/t

The above probabilities may be calculated by calculating the areas under the curve (AUC).

According to the present invention, the discriminatory threshold variation of aspartate may be within the range from 18 to 24 μmol/l, preferentially the discriminatory threshold variation of aspartate is 21 μmol/l.

According to the present invention, the discriminatory threshold variation of taurine may be within the range from 17 to 23 μmol/l, preferentially the discriminatory threshold variation of taurine is 20 μmol/l.

According to the present invention, the discriminatory threshold variation of citrulline is within the range from 22 to 30 μmol/l, preferentially the discriminatory threshold variation of citrulline is 26 μmol/l.

According to the present invention, the discriminatory threshold variation of phenylalanine is within the range from 34 to 46 μmol/l, preferentially the discriminatory threshold variation of phenylalanine is 40 μmol/l.

According to the present invention, the assignment of a score for each marker performed in step d) of the method according to the invention consists of assigning the value 0 or 1 for each marker. The assigned value is dependent on the comparison performed in step c) of the method according to the invention for each marker. If the comparison shows that the absolute value of the result of the calculation performed in step b) is greater than or equal to the discriminatory threshold variation, a score of 1 is assigned. If the absolute value of the result of the calculation performed in step b) is less than the discriminatory threshold variation, a score of 0 is assigned.

Finally, after a score has been assigned for each marker, a sum of said scores is calculated.

According to the present invention, a result of the sum calculated in step d) leads to a score greater than or equal to 2 which indicates transplant rejection.

According to a particular embodiment of the invention, the present invention also comprises a method as defined above wherein the measurement in step a) is performed after transplantation, and wherein the reference concentration in step b) consists of a measurement of the concentration of said marker prior to transplantation.

The experiments conducted by the inventors of the present invention demonstrated that the method according to the invention makes it possible to determine bowel transplant rejection with certainty.

This method offers a very large number of advantages in relation to the methods of the prior art.

Firstly, this method is non-invasive, enabling early diagnosis of transplant rejection without iterative blind, i.e. random, biopsies, performed without any genuine warning signs of rejection, as performed in the prior art. Early detection of rejection makes it possible to increase the survival rate of patients by 11 to 80% through improved medical management.

The non-invasive nature of the method according to the invention also allows better acceptance by the patient of the method according to the invention in relation to iterative bowel biopsies. The non-invasive nature also makes it possible eliminate the risks associated with iterative bowel biopsies with or without bowel endoscopy, such as hemorrhaging, digestive perforation, etc.

The method according to the invention further makes it possible to obtain a 100% reliable diagnosis of transplant rejection unlike the prior art. Indeed, in the prior art, detection is carried out by means of blind biopsy; therefore, the specimen may correspond to a part of the organ not yet showing the histological signs of rejection and therefore lead to an incorrect diagnosis.

Therefore, the method according to the invention enables rapid, reliable and non-invasive detection of bowel transplant rejection and indirectly an increase in the survival rate of transplant patients. It enables early treatment of transplant rejection before it becomes irreversible and results in the loss of the transplant, or even death of the patient.

Further advantages may emerge for those skilled in the art on reading the following examples provided for illustrative and non-limitative purposes.

### EXAMPLES

### Example 1

### Measurement of Marker Concentration in a Fluid Sample

A/ Study Population.

In this example, 24 anaesthetised, intubated and ventilated female Large WithexLandrace pigs weighing between 25 and 30 kg were used. Two groups were studied:

group 1, 8 pigs received a bowel autotransplant, and

group 2, 8 pigs received an allotransplant but with no immunosuppressant treatment.

The subjects from both groups received a segmental bowel transplant of 5±0.2 m after exercise of 70% of the proximal small bowel. All the subjects were sacrificed on the 8^{th} day of the postoperative period.

The survival rate of the subjects on D8 was 100% in both groups.

All the subjects were fed from the first day of the postoperative period ad libitum with piglet complete feed supplied by SAS BERMOND consisting of the following ingredients: wheat, barley, soy bean cakes, small seeds and husks from cereal crops, oilseeds, beetroot pulp, corn, dicalcium phosphate, peas, additive premix, vegetable oil, calcium carbonate, maxid, threonine, lysine, salt, methionine. The contents of the various constituents were as follows: crude protein 17.9%, crude fat 1.3%, crude cellulose 4.4%, crude ash 5.5%, lysine 11.0 g/kg, vitamin A 9000 IU/Kg, vitamin D3 2000 IU/kg, vitamin E 20 IU/kg, copper (in sulphate form) 100 mg/g and avilamycin 30 mg/kg.

The parameters studied at incision (T0) and at the time of sacrifice on the 8^{th} day of the postoperative period (T1) were the amino acid levels in the plasma and the histology of the transplanted bowel.

B/ Sample Preparation

Blood samples were taken from all the subjects prior to transplantation. The samples were taken using a syringe connected to a catheter inserted into the right external jugular vein. The volume of each sample was 4 ml. The plasma fraction and red blood cell separation was performed by means of centrifugation. These samples were the reference samples.

After transplantation, blood samples were taken on the 8^{th} day of the postoperative period. The samples were taken using a syringe connected to a catheter inserted into the right external jugular vein during transplantation. The volume of each sample was 4 ml. The plasma fraction and red blood cell separation was performed as described above. These samples were the test samples. They were frozen at −80° C. pending the amino acid assay.

C/ Marker Concentration Measurement

After sampling before and after transplantation as described above, the citrulline, taurine, aspartate and phenylalanine concentration was measured in each of the samples.

Citrulline, phenylalanine, aspartic acid and taurine were assayed in the biological fluids by means of High Performance Liquid Chromatography (HPLC) coupled with mass spectrometry (LC-MS/MS) as described in the article by PIRAUD Monique et al. “Ion pairing, reversed phase liquid chromatography/electrospray ionization mass spectrometric analysis of 76 underivatized amino acids of biological interest: a new tool for diagnosis of inherited disorders of amino acids metabolism”. Rapid Com Mass Spectrometry, 2005; 19lμ: 1587-1602. [7]

The reagents used for the assay were: HPLC grade methanol HPLC and acetonitrile supplied by CARLO-ERBA (Val de Breuil—France), L-citruline and tridecafluoraheptanoic acid (TDFHA) supplied by SIGMA-ALDRICH (L'ISLE D'ABEAU CHESNES—St QUENTIN FALLAVIER—France). The internal reference standards used were DL-Alanine (2,3,3-D4), the deuterised stable isotope of alanine, phenylalanine (D5 nucleus) and L-aspartic acid (2,3,3-D3), supplied by CAMBRIDGE ISOTOPE LABORATORY (Andover, Mass., USA). Sterile water (Versol-Laboratoires AGUETTANT—LYON—France) was used for preparing the reagents in aqueous solution.

Firstly, the samples were deproteinised by means of High performance Liquid Chromatography in an ethanol medium to assay the free amino acids. By means of an automated sampling and injection system (AGILENT—PARIS—France), the amino acid chromatography applies the principle of ion pairing chromatography. The column has a QS UPTISPHERE 120 Å, 3 μm BP2, 50×2 mm type stationary phase (UP3 BP2#5QS Interchrom, Interchim, Montluçon, France). The gradients applied (water—TDFHA and Acetonitrile) were identical to those disclosed in the article by Monique Piraud et al. [7]

After the separation of the amino acids by means of HPLC chromatography as described above, the paired mass spectrometry analyses were carried out using an API 3000 triple quadrupole mass spectrometer (Sciex Applied Biosystems, Toronto, Canada), the result acquisition software consisting of version 1.4.1. The specific transitions (of non-isotopic amino acids) and those of the reference standards were used in positive mode and the optimal parameters for obtaining same were applied to each molecule. The simultaneous analysis of these transitions made it possible to implement a qualitative and quantitative amino acid analysis method. The quantification value limits for each molecule were compatible with the assay thereof in plasma, urine and cerebrospinal fluid (CSF) by means of prior data calibration with a calibration line for each series of analyses. The analyses were the subject of internal and external (national and international) quality control to verify the conformity of the results obtained and the correct operation of the equipment. The coefficients of variation (CV) of the results obtained were less than or equal to 5% in terms of repeatability and reproducibility.

This method was selected as it enables the assay of 76 amino acids simultaneously with a greater precision, more rapidly (16 minutes of chromatography and mass spectrometry detection) and more economically than the ion exchange column separation method. Indeed, ion exchange column separation is followed by post-column derivation with ninhydrin, this technique requiring a time of 120 minutes of chromatography per sample.

### Example 2

### Determination of Reference Concentration for Each Marker

The reference concentration was determined in two ways during the experiments.

A first method for determining the reference concentration for each subject and for each marker was performed as described in example 1. It consists of taking a fluid sample prior to transplantation (TO) for each subject and measuring, for each marker, the value of the concentration in each sample prior to transplantation.

A second method was used during our experiments so as to determine the reference concentration for each marker. It consists of taking a fluid sample as described in example 1 from a plurality of healthy subjects. After sampling, the concentration of each marker was measured in each sample as described in example 1. The various concentration measurements for each marker were then standardised.

The reference values according to the first method used for the subjects tested in example 1 are given in table 1 below.

TABLE 1
Reference concentrations of each marker for
each subject in example 1

| | | Amino acid | | | |
|---|---|---|---|---|---|
| | | (μmol/l) | | | |
| Subjects | | Citrulline | Taurine | Phenylalanine | Aspartate |
| GROUP 1 | 1 | 59 | 140 | 61 | 21 |
| | 2 | 68 | 179 | 43 | 20 |
| | 3 | 60 | 279 | 78 | 25 |
| | 4 | 87 | 192 | 69 | 14 |
| | 5 | 40 | 278 | 73 | 19 |
| | 6 | 80 | 153 | 79 | 17 |
| | 7 | 108 | 187 | 64 | 23 |
| | 8 | 67 | 194 | 81 | 19 |
| GROUP 2 | 9 | 107 | 102 | 80 | 11 |
| | 10 | 91 | 106 | 56 | 16 |
| | 11 | 111 | 88 | 60 | 11 |
| | 12 | 58 | 79 | 70 | 12 |
| | 13 | 91 | 59 | 54 | 16 |
| | 14 | 62 | 66 | 64 | 13 |
| | 15 | 38 | 41 | 56 | 21 |
| | 16 | 61 | 117 | 67 | 24 |

### Example 3

### Discriminatory Threshold Variation Determination

The discriminatory threshold variations were determined for each marker by defining curves (ROC) constructed on the basis of the various thresholds studied and by establishing the threshold offering the best compromise in terms of sensitivity (Se) and specificity (Sp): maximisation of the Youden index (Se+Sp−1).

1) For each amino acid, the absolute value of the difference between T0 and T1 was calculated: value at T1−value at T0

2) For each variation threshold studied, the sensitivity, specificity, positive and negative predictive value were calculated:

The sensitivity, specificity, positive predictive value and negative predictive value are as defined above.

The plasma levels of four amino acids were significantly correlated via the sensitivity, specificity, positive predictive value and negative predictive value calculations with the incidence of rejection.

The sensitivity of each marker was determined by the probability of the value of the variation between T1 and T0 being greater than the threshold if the animal belongs to the rejection group.

The specificity of each marker was determined by the probability of the value of the variation between T1 and T0 being less than or equal to the threshold if the animal belongs to the non-rejection group.

The positive predictive value of each marker was determined by the probability of the animal belonging to the rejection group if the value of the variation between T1 and T0 is greater than the threshold.

The negative predictive value of each marker was determined by the probability of the animal belonging to the non-rejection group if the value of the variation between T1 and T0 is less than or equal to the threshold.

3) Score Definition

The score was defined on the basis of the variation of a plurality of amino acids, counting 1 point if the value is greater than the threshold and 0 points if the value is less than or equal to the threshold.

In the case of a score constructed on the basis of 4 amino acids (taurine, phenylalanine, aspartate, citrulline), with the following thresholds: taurine: threshold=20; phenylalanine: threshold=40; aspartate: threshold=21; citrulline: threshold=−26:

animal No. i with the values taurine=15; phenylalanine=45; aspartate=25; citrulline=−5: score=3,

animal No. j with the values taurine=15; phenylalanine=32; aspartate=25; citrulline=−35: score=1.

4) Area Under the Curve (AUC) Calculation

A logistic regression model was constructed using SAS software from SAS Institute Inc and described in SAS/STAT Software: “Changes and enhancements through release 6.11”. In. 6.11 ed. Cary, N.C., 10 USA; 1996. [6]. This model comprises the rejection variable as the dependent variable and the score as the independent variable as defined in point 3).

If Y is the response of an animal, annotated 0 in the absence of rejection and 1 in the presence of rejection, x is the independent variable vector and it is the probability of the response to be modelled, the logistic model is expressed according to the following formula:

$$log it ( π ) ≡ log ( \frac{π}{1 - π} ) = α + {β}^{′} x$$

wherein α is the intercept and β′ is the parameter vector.

If N is the total number of observations, t is the total number of response pairs, n_{c} is the number of consistent pairs, n_{d} is the number of discrepant pairs and t−n_{c}−n_{d} is the number of equitable pairs. The area under the curve (AUC) is calculated as follows:

C=(n_{c}+0.5(t−n_{c}−n_{d}))/t

The definition of this curve enabled us to calculate the various statistical values for the implementation of the present invention.

The discriminatory threshold value for each marker, the sensitivity, specificity, positive predictive value and negative predictive value thereof are given in table 2.

TABLE 2
discriminatory threshold variation, sensitivity, specificity, positive
predictive value, negative predictive value for each marker.

| | Discriminatory | | | Positive | Negative |
|---|---|---|---|---|---|
| | threshold | | | Predictive | Predictive |
| Markers | value | Sensitivity | Specificity | Value | Value |
| Taurine | 20 μmol/l | 88% | 88% | 88% | 88% |
| Phenylalanine | 40 μmol/l | 75% | 100% | 100% | 80% |
| Aspartate | 21 μmol/l | 88% | 100% | 100% | 89% |
| Citrulline | 26 μmol/l | 50% | 75% | 67% | 60% |

### Example 4

### Calculation of Difference for Each Marker, Assignment of Score for Each Marker and Summing of Scores

The difference in concentration between the reference concentration of each marker and the concentration after transplantation for the subjects in example 1 was calculated by calculating the absolute value of the difference between T0 and T1: value at T1−value at T0

The concentration for each subject after transplantation is given in table 3.

TABLE 3
concentration for each subject and each marker
after transplantation.

| | | Absolute value of each result | | | |
|---|---|---|---|---|---|
| | | (μmol/l) | | | |
| Subjects | | Citrulline | Taurine | Phenylalanine | Aspartate |
| GROUP 1 | 1 | 37 | 136 | 56 | 21 |
| | 2 | 42 | 186 | 79 | 22 |
| | 3 | 61 | — | — | 43 |
| | 4 | 39 | 187 | 109 | 35 |
| | 5 | 42 | 188 | 63 | 38 |
| | 6 | 35 | 186 | 68 | 34 |
| | 7 | 70 | 185 | 81 | 41 |
| | 8 | 63 | 140 | 60 | — |
| GROUP 2 | 9 | 33 | 1440 | 222 | 480 |
| | 10 | 19 | 502 | 187 | 60 |
| | 11 | 33 | 324 | 100 | 36 |
| | 12 | 25 | 311 | 133 | 56 |
| | 13 | 10 | 109 | 138 | 24 |
| | 14 | 53 | 447 | 166 | 38 |
| | 15 | 16 | 56 | 86 | 45 |
| | 16 | 34 | 276 | 186 | 60 |

— concentration not measured

The score for each subject was assigned according to the comparison for each marker of the absolute values of the difference with the discriminatory concentration. If the absolute value of the difference for a marker was greater than the discriminatory concentration thereof, a score of +1 was assigned. If the absolute value of the difference for a marker was less than the discriminatory concentration thereof, a the score of +0 was assigned. The score was assigned for each subject and for each marker. After score for each marker was assigned, the sum of the scores for each subject was calculated to give the total score.

### Example 5

### Verification of Correlation Between Sum of Scores and Rejection

This experiment was conducted using the subjects in example 3. Bowel histology was performed on a bowel sample with an Olympus BX41 microscope on D8 for each subject in example 1.

The commonly acknowledged classification of bowel rejection is as follows: for the “indeterminate” grade, the lesions are usually minimal and there is an increase in the number of apoptotic cells, but with less than 6 apoptosis per 10 glands and little or no inflammation. For minor rejection (grade 1), the number of apoptotic cells is in excess of 6 per 10 glands and a discrete to moderate predominantly mononuclear inflammatory infiltrate is observed. In moderate rejection (grade 2), an increase in the number of apoptotic cells is observed. The apoptotic cells become confluent and may lead to glandular destruction and a moderate to severe mononuclear infiltrate. Finally, in severe rejection (grade 3), greater glandular destruction with gland depletion zones, or erosions or ulcerations and marked inflammatory infiltrate are observed. These observations are described in the article by Ruiz P et al. “Histological criteria for the identification of acute cellular rejection in human small bowel allografts: results of the pathology workshop at the VIII International Small Bowel Transplant Symposium”. Transplant Proc 2004; 36:335-337 [8].

No anomalies (normal) were detected in the histological observation of the samples from the subjects in example 1 whereas all the subjects in group 2 displayed moderate to severe transplant rejection.

The correlation between the rejection observed histologically and the score calculated was verified as described above.

The breakdown of subjects according to the score obtained and potential transplant rejection is given in table 4.

TABLE 4
Breakdown of subjects according to score obtained.

| | | | | Histological |
|---|---|---|---|---|
| | | | | observation of |
| | | | | transplant |
| Sample | | Group 1 | Group 2 | rejection |
| Score | 4 | 0 | 1 | Yes |
| | 3 | 0 | 4 | Yes |
| | 2 | 0 | 3 | Yes |
| | 1 | 5 | 0 | No |
| | 0 | 3 | 0 | No |
| | Total | 8 | 8 | |

As shown in table 5, it was verified and confirmed that a score greater than or equal to 2 for a subject made it possible to predict the presence of transplant rejection in 100% of cases.

TABLE 5
Breakdown of percentage of subjects according
to score and group

| | | | | Histological |
|---|---|---|---|---|
| | | | | observation of |
| | | | | transplant |
| Percentage | | Group 1 | Group 2 | rejection |
| Score | 4 | 0 | 6.25%* | Yes |
| | | | (100)% | |
| | 3 | 0 | 25%* | Yes |
| | | | (100)% | |
| | 2 | 0 | 18.75%* | Yes |
| | | | (100)% | |
| | 1 | 31.25%* | 0 | No |
| | | (100)% | | |
| | 0 | 18.75%* | 0 | No |
| | | (100)% | | |
| | Total | 8 | 8 | |

*calculation of % with respect to overall total: 5/16 = 31.25%; 3/16 = 18.75%; 1/16 = 6.25%, etc.
calculation of % with respect to row total

### Example 6

### Plasma Citrulline Concentration Study

This example was performed on a population of pigs as in example 1.

As in example 1, two groups were formed:

group 1: 8 pigs received a bowel autotransplant, and

group 2: 8 pigs received an allotransplant but with no immunosuppressant treatment.

A/ Pig Anaesthesia.

The pigs were anaesthetised for transplantation according to the following protocol: intramuscular premedication was administered with 20-25 mg/kg of ketamine (Imalgène 1000, registered trademark, 10 ml, Mérial, Lyon, France), and 0.004-0.01 mg/kg of glycopyrrolate (Robinul V, registered trademark, 5 ml, Vétoquinol, Lure, France); induction with isofluorane gas (AErran, registered trademark, Baxter SA, Maurepas, France) administered with a mask. The 15 animals were intubated using a laryngoscope and an endotracheal tube (Hudson RCI, registered trademark, Sheridan/CF, ID 6.0-7.0 mm) and placed under mechanical ventilation (Veterinary anaesthetic ventilator model 2000, Hallowell FC) (respiratory flow: 15-20/min; F102=30%). During these operations, the pigs were infused intravenously with 50 ml/kg of 0.9% NaCl (B. Braun) and 65 ml/kg 5% glucose (Aguettant isotonic glucose, registered trademark, Aguettant, Lyon, France). Prior to the abdominal incision, a venous catheter was inserted into the external vein to measure the central venous pressure and perform infusion.

The arterial pressure was measured (SC 7000, Siemens, France) via a cervical catheter inserted into the carotid artery.

The heart rate and the oxygen saturation were measured continually using a sphygmo-oxymeter with a detector fixed to a gum. (Model 9847 V, Nonin Medical Inc, Plymouth, UK).

B/ Ischemic Damage Due to In Situ Reperfusion.

The in-situ intestinal ischemia technique used in the present example is derived from that described by Lauronen et al. “Effects of extrinsic denervation with or without ischemia-reperfusion injury on constitutional mucosal characteristics in porcine jejunoileum”. Dig Dis Sci 2001; 46(3):476-85 [9] et de Pakarinen M P, Pirinen P, Lauronen J, Raivio P, Kuusanmaki P, Halttunen J. Effects of transection and extrinsic denervation and a model of autotransplantation of the porcine jejunoileum on cholesterol biodynamics. J Ped Surg 2003; 38(11): 1585-90 [10]. The ileum was cross-sectioned at the proximal end 10 cm from the ileocaecal valve, at the top of the curve formed by the superior mesenteric artery. The corresponding mesentery was sectioned. The superior mesenteric artery was released from the duodenal branch to the second jejunal branch. The jejunum corresponding to the first jejunal artery was sectioned at the first jejunal artery, approximately 30 cm distally from the ligament of Treitz. The corresponding mesentery was sectioned to the origin of the first jejunal artery. All the nerve and lymphatic connections of the jejunum, with the exception of the superior mesenteric vessels, were sectioned. The superior mesenteric artery, the superior mesenteric vein and the colonic branches, from the primary trunk of mesenteric vessels, were skeletised over a length of at least 2 cm (between the origin of the duodenal artery and the second jejunal artery). In this way, the skeletised mesenteric vessels and the colonic branches thereof remained the only connections maintaining blood perfusion of the jejuno-ileum.

After of the intravenous injection of 100 IU/kg of body weight of heparin (Heparin Choay registered trademark), the skeletised mesenteric vessels were clamped to produce jejunal ischemia. The superior [.] was clamped at the proximal origin of the duodenal artery. The duodenal artery and the colonic artery were clamped separately at the origins thereof to prevent the passage of the preservation solution in the systemic circulation while preventing arterial reflux in the jejunal circulation. The superior mesenteric vein was clamped distally from the colonic vein, enabling blood flow via the colonic vein. Subsequently, the superior mesenteric artery was catheterized downstream from the clamp, via the first jejunal artery. The jejunum isolated in this way was washed with 100 ml of cold (4° C.) IGL-1 solution (Institut Georges Lopez, Lyon, France). A small incision of the superior mesenteric vein was performed upstream from the clamp to evacuate the effluent. After cold ischemia, the small incision in the superior mesenteric vein was resealed with a prolene 6-0 suture. The first jejunal artery was ligated at the origin thereof and the vascular clamp was removed, enabling reperfusion of the jejuno-ileum. The jejunal length was then measured along the antimesenteric borders from the proximal end to the distal end. Seventy percent of the proximal jejuno-ileum was resectioned. For this reason, approximately 5 m of the small bowel including 30 cm of the proximal jejunum and 30% of the distal small bowel remained in place. Bowel continuity was restored by means of reanastomosis of the jejunum to the ileum. The mesenteric gaps were then resealed.

C/ Allotransplantion.

In the donor, the superior mesenteric artery and vein were dissected at the colonic branches. The colonic branches were ligated and sectioned. The proximal small bowel was sectioned approximately 30 cm downstream from the ligament of Treitz. The ileum was sectioned 10 cm upstream from the ileocaecal valve, at the top of the curve formed by the superior mesenteric artery. The corresponding mesentery was sectioned. After the intravenous injection of 100 IU/kg of body weight of heparin (Heparin Choay registered trademark), 800 ml/kg of the donor's blood was taken to be used for transfusion to the recipient. The jejuno-ileum isolated in this way was then washed with 100 ml of cold (4° C.) IGL-1 solution via the subrenal aorta. The transplant thus comprised the entire jejunum and ileum, the superior mesenteric artery in continuity with a segment of abdominal aorta, the superior mesenteric vein in continuity with the vena portae. The transplant was stored temporarily in a cold (4° C.) IGL-1 solution. The contents of the small bowel were not washed. After reperfusion, the transplant was reduced from the proximal end of the jejuno-ileal segment, leaving approximately 4.5 m to account for the 30 cm of native proximal jejunum and 10 cm of native ileum left in the recipient.

In the recipient, the proximal small bowel was sectioned approximately 30 cm downstream from the ligament of Treitz and the ileum was sectioned 10 cm upstream from the ileocaecal valve. The isolated ileojejunum was resectioned. The entire colon and rectum of the recipient were retained. The allotransplant was then implanted. The abdominal aorta from the transplant was anastomosed to the subrenal abdominal aorta of the recipient with a prolene 6-0 suture. The superior mesenteric vein was anastomosed to the subrenal inferior vena cava. Digestive continuity was restored by means of anastomosis of the jejunum and the ileum. The mesenteric gaps were resealed. In both groups, the mesentery had a whitish appearance before the abdomen was closed.

D/ Postoperative Treatments.

At the end of the procedure, a single intramuscular dose of 4 mg/kg Tolfenamic acid

(Tolfedine 4%, registered trademark, Vétoquinol, Lure, France) was administered to all the animals for postoperative analgesia and a single intramuscular dose of 15 mg/kg of sustained-action amoxycillin (Clamoxyl L.A. registered trademark, Pfizer) was administered as a preventive antimicrobial treatment. The animals were extubated after the operation and placed in metabolic cages with heating lamps. Each animal received 250 ml of 5% glucose intravenously on the first and second day after the operation using the central venous catheter left in the external jugular vein. The animals were fed from the first day of the postoperative period with feeds as described in example 1. The animals were euthanized on the 8^{th} day of the postoperative period with an intravenous dose of sodium pentobarbital (Dolethal, registered trademark, Vetoquinol SA, Lure, France) after a laparotomy under general anaesthetic with isofluorane.

E/ Results

The survival time, weight, appearance of stools and the bowel 8 days after transplantation were observed as in example 5.

The plasma concentration of citrulline was measured at T0 and T1 as in example 1.

The mean weight measured at the time of transplantation was 25.8±7.6 kg in group 1, and 20.6±2.7 kg in group 2.

The length of the remaining small bowel was 5±0.2 m in both groups.

All the animals survived until the end of the study; therefore, the survival rate on the 8th day was 100%.

A variation in the weight of the pigs in each group was observed:

in group 1, weight loss of up to 10% and weight gain of up to 6% were observed,

in group 2, weight loss of 10 to 15% was observed.

The histological observations on the 8^{th} day after bowel transplantation in group 1 were normal without any sign of rejection.

The histological observations on the 8^{th} day after bowel transplantation in group 2 displayed signs of rejection for all the subjects. The level of transplant rejection was between 3 and 4 for all the subjects in this group. The native small bowel and colon did not display any abnormal histological signs.

The mean plasma concentration of citrulline prior to transplantation and on the 8^{th} day for each group of subjects is represented in table 6 below:

TABLE 6
Mean plasma concentration of citrulline in each of the two groups.

| | Concentration | |
|---|---|---|
| | prior to | Concentration on |
| | transplantation | 8^{th} day |
| Group 1 | 71.1 ± 20.6 | 48.6 ± 13.7 |
| Group 2 | 77.4 ± 26.2 | 27.9 ± 13.5 |
| Comparison of | Non-specific | Significant |
| groups | difference | difference |
| | | (P < 0.01) |

The results given in table 6 describe a significant difference in the plasma concentration of citrulline for the subjects in group 2 on the 8^{th} day. Therefore, the plasma concentration of citrulline is significantly correlated with the incidence of bowel transplant rejection.

Therefore, this concentration has a discriminatory power on the incidence of bowel transplant rejection.

### Example 7

### Application of the Method According to the Invention to Humans

1) Number of Patients

The calculation of the number of patients enrolled, i.e. used in the present test, is based on citrulline as this substance has been associated with the risk of rejection as in example 6 above.

Citrulline was characterised by a lower discriminatory power for the incidence of rejection than that of other markers (taurine, phenylalanine, aspartate), a number of patients based on citrulline with a 20% β risk demonstrates significant associations for the other markers with a sufficient statistical power (at least greater than 80%).

The number of subjects to be enrolled is calculated with 5% bilateral a risk and a 20% β risk and is based on three studies.

The previous study by the author shows a decrease of more than 40% in citrulline in the allotransplanted pigs compared to the autotransplanted pigs where a sample size of 16 was sufficient.

The study by Gondolosi et al. “The value of plasma citrulline to predict mucosal injury in intestinal allografts”. Am J Transplant. 25 2006 Nov. 6(11): 2786-90 [11] conducted on transplanted humans, detected a 40% decrease in citrulline levels in cases of bowel mucosal lesions (23+/−15) compared to patients with no mucosal lesions (38+/−23). On the basis of this hypothesis, selecting a mean standard deviation of 20, a minimum sample size of 60 patients is required to demonstrate such a deviation.

In the study by David A I et al. “An association of lower serum citrulline levels within 30 days of acute rejection in patients following small intestine transplantation.” Transplant Proc. 2006 July-August; 38(6):1731-2 [12], a 35% decrease in the citrulline level was observed in cases of rejection, i.e. 18.8 in a free period versus 12.4 in the month prior to rejection. In this case, the number of subjects required is 30 with a standard deviation of 6.

Therefore, a sample size of 50 patients is chosen for three reasons:

The decrease in citrulline levels may be greater than in the study by Gondolesi et al. as the comparison does not relate to the presence of bowel mucosal lesions or not but to the incidence of rejection or not; therefore the number of patients to be enrolled may be less than 60.

A precaution is taken with respect to the study by David et al. [12] if the difference observed is smaller, a sample size greater than 30 is required.

The groups to be compared in our study (rejection versus non-rejection) do not have identical sample sizes, resulting in a loss of statistical power.

According to the data in the literature, the rejection rate observed is approximately 60%.

2) Application of the Method.

The method according to the invention is applied to a fluid sample from a human selected in the group of fifty subjects as defined above.

The fluid sample is taken by means of a blood sample.

The reference concentration of each marker is that corresponding to the measurement made immediately prior to the bowel transplant or after the transplant, but in the absence of histologically confirmed rejection.

The concentration of each marker is measured using the technique cited above in example 1c).

The difference for each marker is calculated by means of simple subtraction.

The score for each marker is assigned as described in example 4.

The sum of the scores is calculated as described in example 4.

The correlation of the scores with the transplant rejection is performed as described in example 5.

The detection of transplant rejection is observed when the sum of the scores of the markers is greater than 2.

Therefore, the implementation of the method according to the invention makes it possible to detect 100% of cases of bowel transplant rejection as described in example 2 and therefore is suitable for use for the diagnosis of transplant rejection.

### LISTS OF REFERENCES

[1] P. Crenn et al. “Postabsorptive plasma citrulline concentration is a marker of absorptive enterocyte mass and intestinal failure in humans”; Gastroenterology 2000 5; 119: 1496-505.
[2] L C. Lutgens et al. “Monitoring myeloablative therapy-induced small bowel toxicity by serum citrulline concentration: a comparison with sugar permeability tests”. Cancer 2005; 103: 191-9.
[3] P A. Pappas et al. “Serum citrulline and rejection in small bowel transplantation: a preliminary report”. Transplantation 2001; 72: 1212-16.
[4] P A. Pappas et al “An analysis of the association between serum citrulline and acute rejection among 26 recipients of intestinal transplant.” Am J Transplantation 2004; 4: 1124-32
[5] Le Boucher J, et al. “Amino acid determination in biological fluids by automated ion-exchange chromatography: performance of Hitachi L-8500A.” Clinical Chemistry 1997; 43: 1421-28
[6] SAS Institute Inc and described in SAS/STAT Software: “Changes and enhancements through release 6.11”. In. 6.11 ed. Cary, N.C., USA; 25 1996.
[7] PIRAUD Monique et al. “Ion pairing, reversed phase liquid chromatography/electrospray ionization mass spectrometric analysis of 76 underivatized amino acids of biological interest: a new tool for diagnosis of inherited disorders of amino acids metabolism”. Rapid Com Mass Spectrometry, 2005; 19lμ: 1587-1602.
[8] Ruiz P et al. “Histological criteria for the identification of acute cellular rejection in human small bowel allografts: results of the pathology workshop at the VIII International Small Bowel Transplant Symposium”. Transplant Proc 2004; 36: 335-337.
[9] Lauronen et al. “Effects of extrinsic denervation with or without ischemia-reperfusion injury on constitutional mucosal characteristics in porcine jejunoileum”. Dig Dis Sci 2001; 46(3): 476-85.
[10] Pakarinen M P, Pirinen P, Lauronen J, Raivio P, Kuusanmaki P, Halttunen J. “Effects of transection and extrinsic denervation and a model of autotransplantation of the porcine jejunoileum on cholesterol biodynamics”. J Ped Surg 2003; 38(11): 1585-90.
[11] Gondolesi G et al. “The value of plasma citrulline to predict mucosal injury in intestinal allografts”. Am J Transplant. 2006 November; 6(11):2786-90.
[12] David A I et al. “An association of lower serum citrulline levels within 20 30 days of acute rejection in patients following small intestine transplantation.” Transplant Proc. 2006 July-August; 38(6): 1731-2.

1. Method for the in vitro detection of bowel transplant rejection comprising the following steps:
a) measuring the concentration in a fluid sample of at least two markers selected from the group comprising: aspartate, citrulline, taurine and phenylalanine,
b) calculating the difference for each marker of said concentrations measured in step a) with a reference concentration of said marker,
c) comparing for each marker the absolute value of said difference with a discriminatory threshold variation,
d) assigning a score for each marker according to said comparison as follows: if the comparison shows that said absolute value of the results of the calculation performed in step b) is greater than or equal to the discriminatory threshold variation, a score of 1 is assigned, if said absolute value of the result of the calculation performed in step b) is less than the discriminatory threshold variation, a score of 0 is assigned, and summing said scores, a sum greater than 2 indicating transplant rejection.

2. Method according to claim 1, wherein the concentration of at least three of said markers is measured in step a).

3. Method according to claim 1, wherein the concentration of the four markers is measured in step a).

4. Method according to claim 1, wherein the discriminatory threshold variation of aspartate is within the range from 18 to 24 μmol/l, the discriminatory threshold variation of taurine is within the range from 17 to 23 μmol/l, the discriminatory threshold variation of citrulline is within the range from 22 to 30 μmol/l and the discriminatory threshold variation of phenylalanine is within the range from 34 to 46 μmol/l.

5. Method according to claim 1, wherein the measurement in step a) is performed after transplantation, and wherein the reference concentration in step b) consists of a measurement of the concentration of said marker prior to transplantation.

6. Method according to any of claims 1 to 5, wherein the concentration of at least one of the markers is measured using one of the following techniques: High-Performance Liquid Chromatography (HPLC), immunoassay, reversed-phase gas chromatography with pre-column derivation with various reagents, ion exchange chromatography with post-column derivation using OPA and/or ninhydrin, automated ion exchange chromatography using the Hitachi L-8500A device.

7. (canceled)

8. (canceled)

9. Method according to claim 6 wherein said reagents include at least one of 9-fluorenymethylchloroformate, phenylisothiocyanate, dimethylaminaphthalenesulphonyl chloride, dimethylaminoazobenzenesulphonyl chloride and/or 4-fluoro-7-nitrobenzo-2-oxa-1,3-diazole.

10. Method according to claim 2, wherein the measurement in step a) is performed after transplantation, and wherein the reference concentration in step b) consists of a measurement of the concentration of said marker prior to transplantation.

11. Method according to claim 10, wherein the concentration of at least one of the markers is measured using one of the following techniques: High-Performance Liquid Chromatography (HPLC), immunoassay, reversed-phase gas chromatography with pre-column derivation with various reagents ion exchange chromatography with post-column derivation using OPA and/or ninhydrin, automated ion exchange chromatography using the Hitachi L-8500A device.

12. Method according to claim 11 wherein said regrets include at least one of 9-fluorenymethylchloroformate, phenylisothiocyanate, dimethylaminaphthalenesulphonyl chloride, dimethylaminoazobenzenesulphonyl chloride and/or 4-fluoro-7-nitrobenzo-2-oxa-1,3-diazole.
